(19) United States
(12) Patent Application Publication
(10) Pub. No.: US 2020/0221990 A1
(43) Pub. Date: Jul. 16, 2020

(54) SYSTEMS AND METHODS FOR ASSESSING AND EVALUATING RENAL HEALTH DIAGNOSIS, STAGING, AND THERAPY RECOMMENDATION

(71) Applicant: QUADRUS MEDICAL TECHNOLOGIES, INC., New York, NY (US)

(72) Inventors: CAITLYN MARIE CHIOFOLO, New Hyde Park, NY (US); Nicolas Wadih Chbat, Dobbs Ferry, NY (US)

(21) Appl. No.: 16/739,919

(22) Filed: Jan. 10, 2020

Related U.S. Application Data
(60) Provisional application No. 62/930,986, filed on Nov. 5, 2019.
(60) Provisional application No. 62/791,324, filed on Jan. 11, 2019.

(51) Int. Cl. A61B 5/20; G16H 50/30; G06N 3/02; A61B 5/00

(52) U.S. Cl. CPC A61B 5/201; A61B 5/7275; G06N 3/02; G16H 50/30

(57) ABSTRACT

A system for assessing kidney health includes a processing device including an input module configured to receive input values related to kidney function of a patient, and a prediction module having a computation algorithm and/or a model configured to predict a kidney condition and calculate a kidney health score related to at least one of a severity and a probability of the predicted kidney condition, the kidney health score calculated based on the one or more input values. The system also includes an output module configured to present the predicted kidney condition and the kidney health score to a medical professional.

### CROSS REFERENCE TO RELATED APPLICATION

This application claims priority to U.S. Provisional Application No. 62/791,324, filed on Jan. 11, 2019, the content of which is incorporated hereby by reference in its entirety. This application also claims priority to U.S. Provisional Application No. 62/930,986, filed on Nov. 5, 2019, the content of which is incorporated hereby by reference in its entirety.

### BACKGROUND

The present invention generally relates to assessment, diagnosis and evaluation of kidney health. More specifically, the present invention relates to detection, staging, and prediction of kidney conditions, and to therapy recommendations. The present invention also relates to the implementation and use of a processing device or tool for diagnosis, staging and therapy recommendation, and to the display or other provision of kidney health, stage, and therapy recommendation to, e.g., a user (e.g., a patient and/or medical professional).

Kidney conditions, such as acute kidney injury, affect a large number of patients globally. Diagnosis and prediction of kidney conditions is difficult and is affected by a large number of variables. Thus, it can be challenging for physicians to effectively diagnose and treat kidney conditions.

### SUMMARY

An embodiment of a system for assessing kidney health includes a processing device including an input module configured to receive input values related to kidney function of a patient, and a prediction module having a computation algorithm and/or a model configured to predict a kidney condition and calculate a kidney health score related to at least one of a severity and a probability of the predicted kidney condition, the kidney health score calculated based on the one or more input values. The system also includes an output module configured to present the predicted kidney condition and the kidney health score to a medical professional.

An embodiment of a method of assessing kidney health includes receiving, by an input module, input values related to kidney function of a patient, and predicting, by a prediction module comprising a computation algorithm and/or a model, a kidney condition and calculating a kidney health score related to at least one of a severity and a probability of the predicted kidney condition by a prediction module, the kidney health score calculated based on the one or more input values. The method also includes presenting, by an output module, the predicted kidney condition and the kidney health score to a medical professional.

### BRIEF DESCRIPTION OF THE DRAWINGS

The specifics of the exclusive rights described herein are particularly pointed out and distinctly claimed in the claims at the conclusion of the specification. The foregoing and other features and advantages of the embodiments of the invention are apparent from the following detailed description taken in conjunction with the accompanying drawings in which:

FIG. 1 depicts a computer system configured to perform aspects of embodiments of the present invention;

FIG. 2 is a block diagram that depicts an embodiment of a health prediction and analysis system;

FIG. 3 is a functional block diagram depicting aspects of a method of analyzing health data and generating a prediction of a kidney condition; and

FIG. 4 depicts an example of a neural network structure utilized by a kidney health prediction algorithm.

FIG. 5 is a flow chart depicting aspects of acquisition of data related to health information and/or renal health prediction;

FIG. 6 shows an example of small factor data acquisition unit that can perform aspects of methods described herein;

FIG. 7 is a perspective view of the data acquisition unit;

FIG. 8 is a block diagram that depicts an embodiment of a detailed prediction module including a computation algorithm updated by a learning algorithm upon a learning event trigger.

FIG. 9 depicts an application launch portal and view of helpful links;

FIG. 10 depicts an example of a demographics and baseline information view allowing data entry of inputs to renal health calculation;

FIG. 11 depicts an example of a “baseline” information view and a “measurements and stages” view, allowing data entry of inputs to renal health calculation;

FIG. 12 depicts an example of an “Add Measurement” view allowing entry of new measurement information;

FIG. 13 depicts an example of an “Add Measurement” view showing entered measurements prior to calculating a new stage;

FIG. 14 depicts an example of a “Measurements & Stages” view showing an added measurement and resulting stage 0;

FIG. 15 depicts an example of a “Therapy” view showing recommendations for a stage 0 high risk patient;

FIG. 16 depicts an example of a “Therapy” view showing recommendations for a stage 3 high risk patient;

FIG. 17 depicts an example of an “Edit Measurement” view allowing changing of values;

FIG. 18 depicts an example of a “Sort By” view allowing selection of new measurement sort order;

FIG. 19 depicts an example of a “Measurements & Stages” view showing inputs and resulting kidney health stage with corresponding therapy recommendation;

FIG. 20 depicts an example of a “Plot” view showing kidney health stage by different inputs or guidelines in time;

FIG. 21 depicts an example of an “About” view providing general information about application purpose and guidelines;

FIG. 22 depicts an example of a “How To” view providing instructions of how to use the application and each section;

FIG. 23 depicts an example of a “Guidelines” view providing information about the guidelines for staging kidney health or providing therapy recommendation;

FIG. 24 depicts an example of a “Contact” view;

FIG. 25 depicts an example of an “Abbreviations” view;

FIG. 26 depicts an example of a “References” view;

FIG. 27 depicts an example of a “Therapy” view with recommendations ignored;

FIG. 28 depicts an example of a “Therapy” view with recommendation acknowledgment and selection of notification time;

FIG. 29 depicts an example of a “Therapy” view with notification selected and time until next notice shown;

FIG. 30 depicts an example of a view that includes a “Patients” tab showing most recent information of multiple patients;

FIG. 31 depicts an example of the view of FIG. 30, including a “Reports” tab showing analysis of a selected group of patients; and

FIG. 32 depicts an example of the view of FIG. 30, including a “Patients” tab showing predicted or forecasted stage.

### DETAILED DESCRIPTION

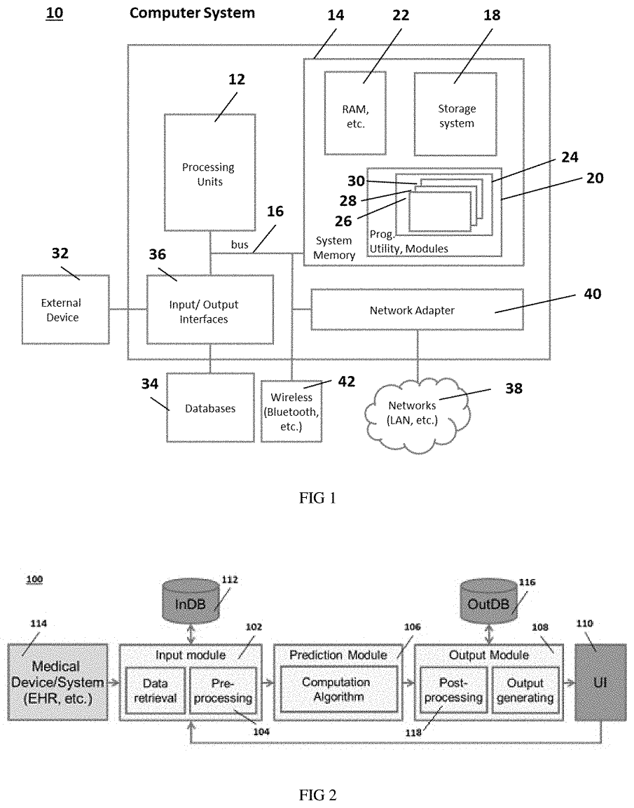

FIG. 1 shows a computer system 10 configured to perform aspects of data acquisition, kidney health evaluation and/or therapy recommendation. In one embodiment, the computer system 10 receives input health data and generates prediction information related to kidney health. The prediction information may include an indication of a predicted kidney condition and a kidney health score indicative of a severity or probability of the predicted kidney condition.

In one or more embodiments, the computer system 10 is configured to perform health evaluation and/or therapy recommendations based on one or more models related to kidney function, health status, treatment outcome, disease risk and/or other information relevant to diagnosis and treatment. Examples of models include inference-based models, artificial intelligence (AI) models, guideline-based, recommendation-based models and others. It is understood that the term model and algorithm may be used interchangeably. Other examples include physiology-driven organ simulation models using a mathematical model of an organ, such as a kidney. A selected model may be a linear or nonlinear model based on clinical variables. The model can output information such as kidney health, disease status and/or therapy recommendations. Further details of the functionality of the computer system 10 are provided below.

Embodiments described herein provide a number of advantages and solutions to problems or challenges faced in diagnosis and treatment of kidney conditions. For example, clinical knowledge, evidence based medicine, and expert opinion may provide rules for diagnosis, staging, and treating kidney conditions, but these rules require some computation of multiple variables or variables in time to be processed, constraints to be applied, and conditions to be checked for proper execution, which can be time intensive and challenging. Embodiments described herein address such challenges, and provide tools that provide such computation and are easily accessible, interpretable, and actionable so as to be clinically useful.

Components of the computer system 10 include one or more processors or processing units 12, a system memory 14, and a bus 16 that couples various system components including the system memory 14 to the one or more processing units 12. The bus 16 represents one or more of any of several types of bus structures, including a memory bus or memory controller, a peripheral bus, an accelerated graphics port, and a processor or local bus using any of a variety of bus architectures. The system memory 14 may include a variety of computer system readable media. Such media can be any available media that is accessible by the one or more processing units 12, and includes both volatile and non-volatile media, removable and non-removable media.

For example, the system memory 14 includes a storage system 18 for reading from and writing to a non-removable, non-volatile memory 20 (e.g., a hard drive). The system memory 14 may also include volatile memory 22, such as random access memory (RAM) and/or cache memory. The computer system 10 can further include other removable/non-removable, volatile/non-volatile computer system storage media.

As will be further depicted and described below, system memory 14 can include at least one program product having a set (e.g., at least one) of program modules that are configured to carry out the functions of embodiments of the invention.

For example, the system memory 14 stores a program/utility 24, having a set (at least one) of program modules. The program/utility 24 may be an operating system, one or more application programs, other program modules, and program data. The program modules generally carry out the functions and/or methodologies of embodiments of the invention as described herein.

For example, the program modules include an input module 26 configured to acquire data such as patient data that can be used as input to a disease detection, staging, and/or prediction model. The program modules can also include a prediction module or evaluation module 28 configured to generate a prediction of kidney (or other organ) disease severity or probability using a prediction model, and an output module 30 configured to output information such as prediction of kidney injury and/or therapy recommendations based on predicted kidney injury, probability, or severity.

The one or more processing units 12 can also communicate with one or more external devices 32 such as a keyboard, a pointing device, a display, and/or any devices (e.g., network card, modem, etc.) that enable the one or more processing units 12 to communicate with one or more other computing devices. In addition, the one or more processing units 12 can communicate with an external storage device such as a database 34. This database may be a data repository of a hospital system, an electronic health record, a medical device or system with proprietary storage, or the like. Such communication can occur via Input/Output (I/O) interfaces 36. Other interfaces might include application programming interfaces (APIs) not shown here.

The one or more processing units 12 can also communicate with one or more networks 38 such as a local area network (LAN), a general wide area network (WAN), and/or a public network (e.g., the Internet) via network adapter 40. The processing units 12 can also communicate wirelessly via, for example, a Bluetooth connection 42 or the like. It should be understood that although not shown, other hardware and/or software components could be used in conjunction with the computing system 10. Examples, include, but are not limited to microcode, device drivers, redundant processing units, external disk drive arrays, RAID systems, tape drives, and data archival storage systems, etc.

FIG. 2 illustrates an example of a system 100 for acquiring health data, analyzing the health data and providing predictions and/or assessments of kidney health and/or therapy recommendations. Aspects of the system 100 may be incorporated in the computer system 10 of FIG. 1, or any other device or system capable of analyzing health data. The system 100 provides a platform for assessing kidney health, which includes analyzing health data to calculate kidney health and/or predict a kidney condition. A kidney condition refers to any disease, condition, or level of kidney function associated with non-optimal kidney function or kidney function below a desired level. Examples of kidney conditions include acute kidney injury (AKI), reversible kidney damage, irreversible kidney damage, recurrent kidney injury, acute kidney disease, intrinsic kidney disease, extrinsic kidney disease, intrarenal renal conditions, pre-renal conditions, and post-renal conditions. The system 100 is configured to receive inputs, which may be measured or known inputs and estimated inputs. As described herein, “inputs” or “input data” includes health data relating to a specific patient and/or other patients, and any other data that can be used to predict a kidney condition and/or assess kidney function.

The system 100 is configured to generate a prediction of a kidney condition, which includes an indication of a predicted kidney condition and may include a kidney health score related to a severity, probability or indicator of the predicted kidney condition. In one embodiment, the prediction is accompanied by an indication of a level of confidence that the predicted kidney condition and/or the kidney health score are accurate. The level of confidence may be presented as a numerical score, a percentage, a probability (e.g., a probability score and/or probability distribution), a visual indicator (e.g., traffic light) and/or any other indication of a level of confidence. The level of confidence may be for a single prediction or multiple predictions. For example, a predicted kidney condition (e.g., an acute kidney injury or AKI stage) includes multiple predictions, each of which is associated with a kidney health score and/or level of confidence and/or probability. Each prediction can be presented to a user, or only the most likely prediction (and its confidence and/or probability) can be presented.

The system 100 is configured to output kidney health prediction information, which includes the predicted kidney condition, kidney health score and/or level of confidence, and may also provide additional information and/or guidance. For example, the prediction information can include a diagnostic protocol for diagnosing the predicted kidney condition, a recommendation of one or more diagnostic tests for evaluating kidney function, a treatment protocol for treating the predicted kidney condition, a therapy recommendation, and/or a recommendation as to an adjustment of an existing treatment protocol. The prediction information may be output to, e.g., a user interface, a processor and/or a storage device. For example, the predicted kidney condition and the kidney health score are output to a medical professional and/or a storage location accessible by a medical professional, and/or directly or indirectly communicated to a medical professional. Aspects of the system 100 and methods for evaluating or assessing kidney health are discussed in more detail below.

Referring again to FIG. 2, the system 100 includes an input module 102, a prediction module 106 and an output module 108. It may further comprise a pre-processing 104 and post-processing module 118. It is noted that the system 100 is not limited to that shown in FIG. 2, as the system 100 may have fewer components or modules than shown in FIG. 2, or may have additional components or modules, or otherwise have any suitable configuration.

The input module 102 can receive input data in a number of ways. For example, inputs can be entered manually, e.g., via a user interface 110, or automatically imported or auto-entered from a file or other memory location. For example, inputs can be entered and/or retrieved (via, e.g., Bluetooth, internet, etc.) from an input database 112 (also referred to as a health information database), from an electronic charting application (as electronic health records or EHRs 114), a lab system, a computerized physician order entry (CPOE) system, interface engine (upon updated or new values), or other medical device/system with a structured data type (e.g. FHIR, HL7, xml, binary, etc.).

Input data may be pre-processed, for example, by the pre-processing module 104. The pre-processing module 104 may be incorporated into the input module 102 as shown in FIG. 2, or be a separate module. Pre-processing may include filtering input data, removing outliers, detrending, normalizing, imputing, time synchronizing, unit conversion, data or file format conversion, and others. Filtering can include, for example, windowing, calculating statistical moments or measures, kernel filtering, etc.

The system 100 may also include user authorization, authentication, and data encryption modules necessary for protecting user and patient privacy (not shown here).

In some embodiments, the input module 102 can be configured to retrieve and automatically input/enter data through integration with hospital systems and devices, including but not limited to the electronic health record (EHR) 114 shown in FIG. 2. In such embodiments, a data retrieval system that can receive, parse, analyze, interpret, and send responses via various industry standard or manufacturer proprietary communication protocols or APIs (HL7, FHIR, binary, or ASCII messaging, etc.). In these embodiments, a user can edit and update prepopulated data or a user may be restricted to read-only view. In these embodiments, manual data entry described herein throughout may be replaced with automatically entered or charted information. It is noted that some data entry can be automatic and other entry can be manual by a user.

In one embodiment, a data acquisition system can be used to acquire various information. For example, data can be automatically electronically extracted from medical devices and/or systems. This extraction may be performed via an API, RS232 communication and/or other mechanisms.

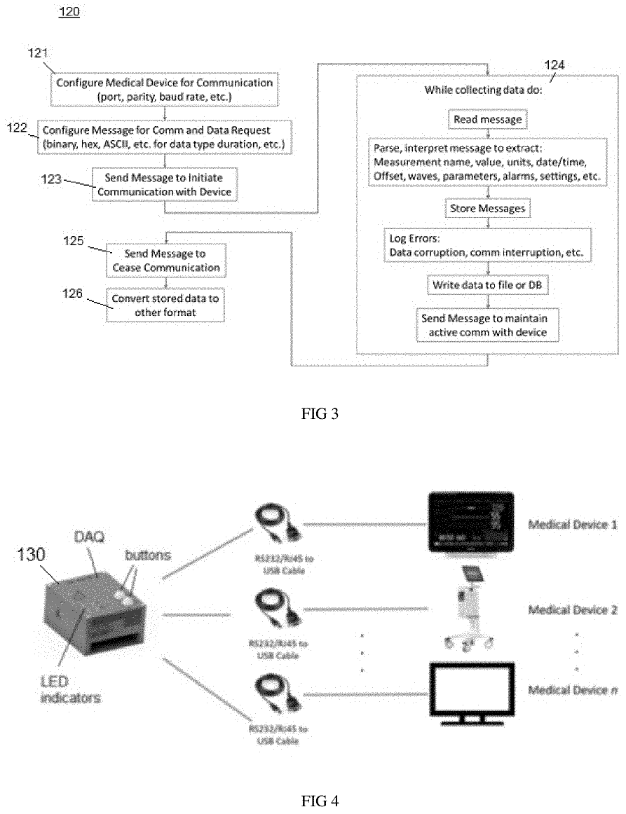

FIG. 3 is a flow chart showing an example of a data acquisition method 120. The data acquisition method 120 utilizes software for performing renal evaluation and recommendation, as well as any other functions described herein. The software may be included in a product that includes the software, which is referred to herein as a “renal health application.” The renal health application may be a mobile application, a desktop program, etc. The method can be performed by a processor, which may be same processor that executes the renal health application or may be a separate processor. The method 120 may be executed once the devices (from which to acquire data) are selected.

The data acquisition system in this example sends and receives messages to establish communication with medical devices and systems, receives information or messages containing information and/or data, parses or processes the data according to standard or proprietary protocols, and stores the data in a repository accessible by the application. Data types and protocols may be of any suitable type and may include one or more of those described herein. The parsing of the data can include separating data elements and attributes, such as numerical value, string value, units of measure, date, time, or datetime stamp, etc. The processing of data can include scaling, normalizing, filtering, unit conversion, etc. The storage involves storing to, for example, a binary file, database, comma separated value (CSV) file, or other such container. Alternatively, or additionally, the data acquisition system can be used to send messages containing data or information to a clinical decision support application for near or real-time remote monitoring and diagnostic or therapeutic decision support.

The method 120 includes a number of steps or stages represented by blocks 151-156. The method 150 may include all of the steps or stages in the order discussed, may include fewer than all of the steps or stages, or may include additional steps or stages not shown.

At block 121, a medical device is configured for communication, and at block 122, a message is configured by the data acquisition system for communication and/or data request purposes. The message is then sent to initiate communication with the medical device (block 123). At block 124, the data acquisition system collects data from the medical device and may perform various other functions, such as reading and storing messages, parsing and processing data collected from messages, logging errors, storing data (e.g., in a file or database) and sending messages to maintain active communication with the medical device. Once data collection is complete, the data acquisition system sends a message to cease communication with the medical device (block 125) and may also convert stored data to other formats as desired (block 126).

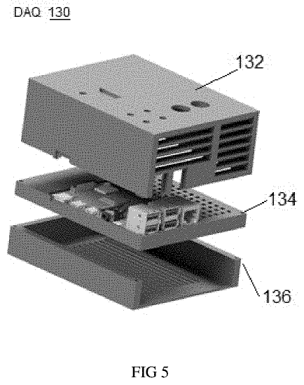

FIG. 4 shows an example of components of a data acquisition system, which includes a data acquisition (DAQ) unit 130 in a small factor format, which includes buttons for prompting data acquisition. The DAQ unit may include LED lights or other indicators that indicate the status of data acquisition from different medical devices, as well as the status of the DAQ software. For example, the DAQ unit is connected to any number n of medical devices, e.g., devices shown in FIG. 4 as Medical Device 1, Medical Device 2 and Medical Device n.

FIG. 5 is an exploded view of the DAQ unit 130. As shown, the DAQ unit 130 includes at least a housing 132, an electronics bay 134 and a bottom bay 136 for a hard drive or other component.

In some embodiments, the data acquisition system can be software that runs on a PC or server, for instance as part of the input 102 and pre-processing 104 modules of FIG. 2. In other embodiments, the data acquisition system can be a small form factor hardware device that is housed in a patient room in a care unit of a hospital and connects to devices in that room. The connection to systems and devices can be wired (LAN, serial, etc.) or wireless (Bluetooth, WLAN, etc.), for instance as part of the wireless 42 and networks 38 blocks of FIG. 1.

Referring again to FIG. 2, the prediction module 106 is configured to analyze the input data to generate a prediction of a kidney condition and/or a kidney health score. For example, as discussed further below, the prediction module 106 predicts a condition such as a kidney injury and provides a kidney health score corresponding to an intrinsic renal injury severity. For example, as discussed further below, the prediction module 106 predicts a condition such as an intrinsic renal injury and provides a kidney health score indicating its probability and/or severity. For example, as discussed further below, the prediction module 106 predicts a condition such as an acute kidney injury (AKI) and provides a kidney health score corresponding to an AKI severity or stage. The prediction may be a single prediction and score (e.g., intrinsic injury, with a probability score of about 30%) or multiple predictions (e.g., prerenal, 20% probability; and intrinsic renal, 70% probability; and postrenal, 10% probability; and no renal injury, 0% probability; or stage 1, 10%; and stage 2, 20%; and stage 3, 65%; and stage 0, 5%).

The output module 108 generates output data that can be sent directly to a user (e.g., a physician or patient) via the user interface 110 and/or stored or archived. For example, kidney health prediction information can be stored in a results database 116, exported manually or automatically, and/or rendered for display on an end-user device (e.g., a smartphone, computer, tablet, web browser). Output data may be sent to an electronic charting application (EHR), lab system, CPOE system, interface engine or any other suitable device, system or location. The output module may also send data to a user via e-mail, SMS message, or the like, e.g., via the network adapter 40.

In some embodiments, the system 100 may also include a post-processing module 118. The post-processing module 118 may be incorporated into the output module 108 as shown in FIG. 2, or be a separate module. The post-processing module 118 sends or otherwise provides prediction module outputs to the output module 108 and is configured to perform at least one of thresholding, scaling, normalizing, converting to a probability, computing a level of confidence, or performing an inference on the one or more prediction module outputs to obtain the predicted kidney condition, its score, probability, indicator, and/or its level of confidence.

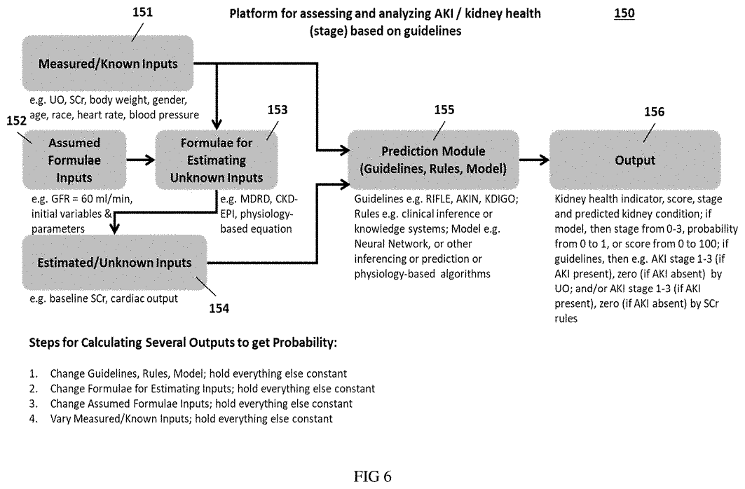

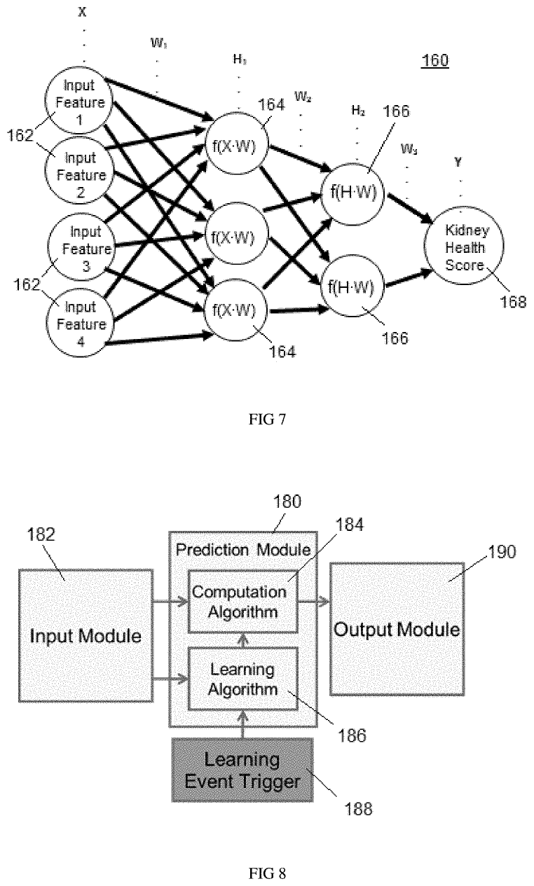

FIG. 6 is a block diagram showing aspects of a prediction and analysis method 150 that can be performed by or with a computing or processing device such as the computer system 10 and/or the system 100. The method 150 includes a number of steps or stages represented by blocks 151-156. The method 150 may include all of the steps or stages in the order discussed, may include fewer than all of the steps or stages, or may include additional steps or stages not shown.

At block 151, the input module 102 receives health data, which may include measured and/or known input data. For example, the system 100 can utilize already existing health data for a patient, such as data typically collected by a routinely used device or sensor, by a medical device, sensor, or system in a hospital or by a physician, nurse, or other care provider, and can thus be performed in some instances without any new or invasive data collection.

Measured and/or known input data includes vitals, demographic data, lab data and other data collected from a patient and/or from similar patients. Examples of vitals include blood pressure (BP), respiratory rate (RR), heart rate (HR), and blood oxygen concentration (SpO2). Examples of demographic data include age, gender, weight and medical history, and examples of lab data include serum creatinine (SCr) levels, sodium (Na) levels, urea nitrogen levels and others. Other measured and/or known input data includes medication information, dialysis information, fluids intake and output (e.g. urine output (UO)), family history, comorbidities or chronic conditions, procedure or test results, other scores, etc. It is noted that the above examples are not intended to limit the number or type of known and/or measurement data.

In addition to measured and/or known input data, the input data can include estimations of unknown data values (i.e., estimated input data). For avoidance of doubt, estimations herein can be interchangeably used with calculated or computed data. Estimated input data includes data values that are not previously known or measured, but are instead calculated or estimated based on known information.

For example, at block 152, the pre-processing module 104 or the prediction module 106 of system 100 defines assumed inputs for use in estimating unknown input values. The assumed inputs can be applied to various formulae (block 153) to generate estimated inputs (block 154).

Estimated inputs include, e.g., estimated lab results, vital signs and fluid measurements. For example, an assumed glomerular filtration rate (GFR) can be used to estimate baseline or estimated serum creatinine (SCr) levels. Various formulae can be used to derive estimated inputs, such as Modification of Diet in Renal Disease (MDRD) equations and Chronic Kidney Disease-Epidemiology Collaboration (CKD-EPI) equations. Other physiology-based ordinary or partial differential dynamic equations can also be used to derive estimated inputs.

At block 155, the input data (including measured and/or known input data and/or estimated input data) is sent to the prediction module 106, which predicts a kidney condition and optionally generates a severity or probability score based on one or more guidelines, rules and/or models (referred to herein collectively as “models”).

The models may include any guidelines, formulae, rules, models or algorithms that enable a prediction of the kidney condition. Examples of such models also include physiology, correlation, time series, nonlinear input to output mappers, algebraic equations, first principle models, deterministic and/or stochastic models, and/or inference systems based on clinical or inferred rules. Examples of such models include various clinical guidelines for detection and/or staging of disease, such as Kidney Disease Improving Global Outcomes (KDIGO) criteria, Acute Kidney Injury Network (AKIN) criteria, and/or Risk Injury Failure Loss End-Stage (RIFLE) criteria. The models may include formulae such as formulae for computing baseline serum creatinine or baseline glomerular filtration rate (e.g. MDRD and CKD-EPI).

At block 156, the output module 108 outputs kidney health prediction information, which includes a prediction of a kidney condition and/or a kidney health (severity) score. The output module 108 is configured to provide various information including the result of applying the input data to the guidelines, rules and/or models.

The health prediction information includes an indication or description of the predicted kidney condition, and may also include an indication of a level of confidence of the predicted kidney condition. The level of confidence may be associated with a single prediction of a kidney condition, or multiple predictions of the kidney condition.

For example, the predicted kidney condition is a prediction that the patient has an Acute Kidney Injury (AKI) and the severity score is an AKI stage (i.e., stage 1-3, or 0 for no AKI stage). However, embodiments described herein are not so limited. For example, the predicted kidney condition can be an AKI, reversible kidney damage, (and, e.g., a score indicating the level of damage), irreversible kidney damage, recurrent kidney injury, acute kidney disease, intrinsic kidney disease, or extrinsic kidney disease. It is noted that a prediction of whether a kidney disease is extrinsic or intrinsic, or whether the kidney disease is reversible or not enables the prediction to be associated with an actionable therapeutic response. For example, as discussed further below, a predicted kidney condition may be output with enough specificity (e.g., including details related to extrinsic vs. intrinsic and/or details related to reversible vs. non-reversible) to allow a user to readily identify an appropriate treatment or therapy.

In one embodiment, the prediction is a single prediction, e.g., a single AKI stage prediction. The AKI stage prediction can be associated with an indication of a level of confidence in the form of a confidence interval or score.

The confidence score can be calculated in various ways. For example, the confidence score can be calculated based on one or more of the following metrics:
a ratio of known and/or measured inputs to a total number of inputs. For example, the ratio is equal to the number of known and/or measured inputs divided by the total number of inputs (known and/or measured inputs+estimated inputs);
a confidence score corresponding to a distance or proximity of an input to an invalid value or range (where an input close to an invalid range or value is considered a low confidence and an input farther way is considered a higher confidence);
a confidence score corresponding to a time since last measurement, or a distance or proximity of an input value's measurement time to the current time (where, for example, a measurement time farther from the current time is considered a lower confidence and a measurement time closer to the current time is considered a higher confidence);
a sum of a plurality of confidence scores (e.g., weights) for different inputs;
an average of confidence scores or weights for different inputs or other statistical attributes;
a confidence score (e.g. 95%) on an output health score (e.g., AKI stage) based on the confidence scores (e.g. 95%) for each input;
a confidence score (e.g. 95%) on an output health score (AKI stage) based on a population's outputs or an individual's historical outputs (e.g. mean+/−1.96*std dev); and
a combination of one or more of the aforementioned metrics.

In one embodiment, the indication of a level of confidence is based on generating a plurality of predictions and providing a confidence score (e.g. including probability) indicating a level of confidence of each prediction. For example, multiple predictions may be generated by re-computing or re-deriving estimated inputs using different formulae and/or using different assumed input values. In another example, multiple predictions are generated by re-sampling assumed or baseline inputs, and/or by re-calculating the prediction using different models or using different input values to the same model(s). In another example, multiple predictions are generated by random perturbation to known inputs and re-calculating the prediction using different input values to the same model(s). Random selections and/or perturbations may be selected using any of various approaches and simulations, such as a Monte Carlo-like simulation or bootstrapping algorithm. Examples of values that can be perturbed or selected include one or more input values, a guideline, a rule, a model used for estimating outputs, and a formula or a model used for estimating inputs.

For example, a predicted AKI stage includes multiple predictions, each of which is associated with a level of confidence and/or probability. Each prediction can be presented to a user, or only the most likely prediction (and its confidence and/or probability) can be presented.

For example, the prediction module 106 receives health data for a patient that includes both urine output (UO) and serum creatinine (SCr). The prediction module 106 generates a first prediction that includes an AKI stage calculated by applying UO values to selected models (e.g., RIFLE, AKIN, KDIGO, and/or a neural network or other model developed/trained to predict AKI stage). The prediction module 106 also generates a second prediction that includes an AKI stage calculated by applying SCr values to the same models. A confidence score is generated for each prediction, and both predictions with their associated confidence scores are presented to a user via the user interface 110, or only the most likely prediction (the prediction associated with the highest confidence score) is presented.

Different predictions can be generated by using a different combination of models and/or inputs and/or assumed inputs and/or estimated inputs. For example, multiple predictions (e.g., AKI stages) can be derived for a given time or time frame by running measured and/or known inputs and estimated inputs through alternative models. In another example, assumed inputs are run through different formulae used for estimated inputs in order to get different estimated inputs (re-estimated inputs), and each set of estimated inputs is applied to selected models to get different outputs. In another example, different input values are selected (for example, at random from within the 95% confidence interval of that input) and those randomly sampled inputs are applied to the same models to get different outputs.

The following are additional examples of re-estimated inputs:
outputs of a different formula (or model) given the same inputs (e.g., estimated GFR from MDRD vs. estimated GFR from CKD-EPI vs. estimated GFR from a physiological model pertaining to an individual);
outputs of the same formula (or model) given varied/resampled inputs (estimated SCr from MDRD assuming a first selected baseline GFR (e.g., 60 ml/min) as compared to estimated SCr assuming a second selected baseline GFR (e.g., 70 ml/min);
outputs of the same physiology model pertaining to an individual given varied/resampled baseline input variables (e.g., calculated SCr from MDRD assuming a first selected baseline GFR (e.g., 60 ml/min) as compared to calculated SCr assuming a second selected baseline GFR (e.g., 70 ml/min); and
outputs of a different model (e.g. guidelines, rules, models) given the same inputs (e.g., AKI stage based on RIFLE vs. AKI stage based on AKIN vs. AKI stage based on KDIGO vs. AKI stage based on a neural network or other algorithm developed/trained to predict AKI stage).
   Examples of different input values include multiple values of inputs provided as an input value distribution to generate a distribution of output values. Input values can be selected from a random distribution of values. For example, multiple values of calculated SCr are calculated from MDRD assuming GFR sampled from normal distribution (e.g., with mean 90 and std dev 30). In another example, multiple values of SCr are sampled from a selected distribution (e.g., a normal distribution with mean 1.0 and std dev 1.0, with right skew). Another example is a selection of body weights from a selected distribution (e.g., a normal distribution with mean 70 and std dev 10). The different input values may be selected as the upper and lower bounds of a confidence interval (e.g., 95%). For example, upper and lower values for body weight are selected as (50.4, 89.6); mean 70±1.96*std dev 10.

In one embodiment, where multiple outputs are generated (by re-processing or re-sampling), the confidence score may be selected based on a population's outputs, an individual's historical outputs or the individual's current inputs (given re-processed/re-sampled inputs to get multiple outputs at a single time point).

Referring again to FIG. 2, in one embodiment, the system 100 is configured to execute an algorithm that, based on input data (e.g., hospital data records, clinical and demographic data), predicts a kidney condition, as well as a score or indication of the severity of the kidney condition. The algorithm is referred to herein as a prediction or computation algorithm.

The prediction algorithm can utilize various input data to predict the condition. For example, input data can be applied to the prediction algorithm in real time or otherwise as new data becomes available. For example, the prediction algorithm is executed as frequently as ICU (intensive care unit) data or other data (e.g., electronic health record (EHR) data) is updated or new data is available. Input data can be sent to the prediction algorithm on a timed-basis, on user request for new/updated scores, on user action at a user interface, and/or on an event-basis (e.g. when the most or least frequently measured input of all the inputs needed for the prediction algorithm is recorded).

The prediction algorithm maps inputs to a kidney health score, such as an AKI score (e.g., stage number) or a probability. Other severity scores may include, e.g., percentage of kidney function, a number score from a selected range (e.g., 0 to 100), a visual indicator showing severity by color or shade (such as a heat map or a traffic light display), or any other visual indicator (e.g., a dial, like a car speedometer with colored regions and a needle indicator).

For example, the kidney health score can be portrayed via a traffic light pattern where green is very low or no risk of AKI, yellow is an intermediate risk of AKI and red is high risk of AKI. Thresholds of AKI risk can be based on population studies, which can be periodically updated (e.g., every week from a selected location or region such as hospital location or geographic region), or updated after data has been collected from a number of patients.

Outputs that can be generated by the prediction algorithm include a) a likelihood of kidney failure, b) an assessment of the nature of the kidney condition (e.g., whether pre-renal, intra-renal or post-renal, and/or whether intrinsic or extrinsic), c) a severity of the kidney condition (e.g. stage or probability), d) whether the kidney condition is reversible (or not) or likely to be reversed with minimal intervention (e.g. fluid-responsive vs. tubular damage requiring more intervention); and/or e) the length of time it will take for the kidneys to enter the predicted condition.

The prediction algorithm produces a kidney health score as an indication the predicted health of the kidney function of a specific patient. The prediction algorithm can produce a kidney health score at any desired time or with any desired frequency. For example, a kidney health score can be output in real time at every instance that new input data (of that patient) is presented.

Mathematically, the prediction algorithm maps health data from the input module 102 to the output module 108 based on a nonlinear correlation between input values and kidney conditions and/or scores. The correlation may be built/trained using previously collected patient data (e.g., EHR for a given patient and/or data regarding similar patients).

Examples of models or algorithms that provide the above-mentioned correlation include neural network (shallow or deep), nonlinear regression (logistical, polynomial, etc.), inference engine (fuzzy, neuro-fuzzy, support vector machine, etc.), and genetic algorithm (GA) clustering. The prediction algorithm is not so limited and can provide the correlation in any of various ways, including other machine learning, data mining and/or artificial intelligence (AI) approaches.

FIG. 7 illustrates an example of a prediction algorithm that employs a neural network structure 160. In this example, the neural network structure includes four input features (forming an input layer), two intermediate (or hidden) layers, and one output neuron that outputs a kidney health score.

Each neuron 162 in the input layer represents an input value shown as a value X (e.g., an assumed, estimated, or measured, or known input such as UO, SCr, body weight, GFR). One value is output from each neuron 162, and each output is given a weight coefficient (or simply a weight). The weighted sum of each output (represented by an arrow) forms an input to each neuron 164 in a first intermediate (hidden) layer H. Each neuron 164 is a computational unit represented by a mathematical function that computes a value by processing its input through a math function (ƒ). The sum of the weighted outputs of the math function (ƒ) of neurons 164 are applied as inputs to neurons 166, each of which is a computational unit represented by a mathematical function (ƒ). Examples of ƒ are sigmoid, hyperbolic tangent, softmax, rectified linear unit (ReLU), leaky ReLU, parameterized ReLU, etc.

Functions ƒ can be the same or different. In this example, the arguments of functions ƒ represent weighted sums of their inputs. X is the vector of input neurons 162, X·W is a dot product of the vector X, W is a weight vector (W₁ in this example), and ƒ(X·W) is a function of this dot product. H is an output of the neurons 164 (H₁ in this example), ƒ(H·W) is a function of the dot product of the vector H and a weight vector W (W₂ in this example), and lastly Y is the output (value, vector, etc.) of the Neural Network. The output Y can be the weighted sum of outputs from the neurons 166 as applied to neuron 168, which calculates a kidney health score. In the example of FIG. 4, there is one input layer and two intermediate layers, and three weight vectors W₁, W₂ and W₃. Embodiments described herein are not so limited, as there can be any number of layers (and any number of corresponding weight values).

The Neural Network structure can be shallow (no or single hidden layer) or deep (two or more hidden layers), fully or partially connected, recurrent, convolutional, adaptive, tap-delay, etc. The network could exhibit feedforward, feedback, lateral, reflexive, or gated, or other connections. Other types of linear or nonlinear mappers (between input and output) can also be utilized.

The neural network may be used periodically, in real time as new data comes in, or otherwise. For example, every time an input patient data record is presented a forward computation produces a kidney health score. This can be run for a single patient or for multiple patients in parallel.

The prediction algorithm may be developed or trained using a variety of mathematical approaches. Development of the prediction algorithm is based on an understanding of the clinical problem, for example, knowledge of patient's symptoms and/or measurements and their general relationship to kidney conditions and kidney function. In one embodiment, input data is collected retrospectively from a patient, other healthy patients (e.g., of similar demographics) and patients having kidney conditions with corresponding disease information via annotation or otherwise.

Relevant input data (clinical, demographic, physiological, lab results, etc.) is selected using univariate analysis and/or other methods to test the power of predictability of the kidney condition using an individual input feature. Relevant input data, or a set of relevant input data, may also be selected using multivariate analysis and/or other methods to test the power of predictability of the kidney conditions using a set of input features.

If a neural network is employed, a random set of weight coefficients for each neuron may be initially selected. An iterative optimization algorithm (which may be or include a learning algorithm) may be used to update the values of the weight coefficients every time an input data set with corresponding disease information is presented.

An iterative optimization algorithm can be steepest descent (back propagation, with or without momentum learning), any gradient-based (1st or 2nd order) optimization algorithm such as: Newton's, Davidon-Fletcher-Powell, Broyden-Fletcher-Goldfarb-Shannon, Conjugate Gradients, etc., any gradient-free (zero-order) optimization algorithm such as: Powell, Zangwill, Hooke-Jeeves, etc., or others with or without momentum learning (e.g. stochastic gradient descent, Adam, Nadam, etc.).

A supervised learning algorithmic approach, using, for example, a computational/prediction algorithm like a neural network, uses collected healthy and sick patients' data for training. Training is accomplished before the prediction algorithm can be useful. Training generally includes using collected data (from healthy and sick patients) in order to compute a set of weight coefficients iteratively, typically, until one or more accuracy criteria are met. When these weights are computed, the neural network can be used for prediction.

Once the optimization algorithm is complete, a final set of weight coefficients is set, and the (trained) computation/prediction algorithm is ready to be used to generate prediction information regarding a kidney condition and/or kidney health score (e.g., a severity and/or probability score).

In one embodiment, the computation/prediction algorithm can be run in a static mode where coefficients are kept constant as new data is received and the prediction algorithm is repeatedly executed. In another embodiment, shown in FIG. 8, the computation/prediction algorithm includes a learning capability that causes the weight coefficients to be updated as new patient information becomes available. As described herein, “learning” refers to updating weight coefficients based on new data being collected (e.g., data from new patients).

For example, a prediction module 180 receives input data from an input module 182. The prediction module may be, for example, included in the prediction/evaluation module 28 and/or the prediction module 155. In this example, the prediction module executes a computation algorithm 184 and a learning algorithm 186 that is prompted or triggered by a learning event 188. The prediction module 180 outputs data to an output module 190, e.g., sends data to a medical professional.

The learning portion of the prediction algorithm may use similar optimization techniques as those described above, but with updated data. The updates can be updated using the learning portion at various times and intervals. For example, the learning can be performed at different schedules, spanning different time periods, and/or for different patient groups.

Learning steps, in one embodiment, are as follows: 1) define the schedule at which to update, 2) define the time period over which to retrieve patient data to be used in the update, 3) define the patient group from data is retrieved, 4) input new patient data to the optimization algorithm, and 5) update weight coefficients. Times and/or schedules at which to update include, e.g., daily, weekly, monthly, yearly, and time periods over which to retrieve data include, e.g., weekly, monthly, yearly. Patient groups can be selected from, e.g., single units (ICU), multiple units (ICUs), multiple hospitals and multiple geographical locations (e.g., multi-states or countries vs. singular). With reference to FIG. 8, the selected update time, interval, and schedule, the availability of new or updated patient data, the availability of data from new patient groups, or user request (e.g. button push, etc.) to update the algorithm are all examples of triggers for a learning event 188.

An example of developing the prediction algorithm and generating a prediction is discussed as follows with reference to FIG. 7. In this example, a nonlinear input-to-output mapper configured as the neural network 160 is used to generate predictions.

Initially, a structure, such as the neural network 160, is selected. In this example, the neural network 160 includes an input layer X that includes neurons 162 representing a number p of input values (e.g., BP, RR, HR, etc. from a patient). An output layer Y includes one or more neurons 168, where each neuron 168 provides an output value (e.g., health score of a disease, a calculated physiological variable like kidney health, etc.). A number N of intermediate, or hidden, layers H provide the structure of this mathematical network. Each hidden layer has a number of neurons. For example, layer H₁ includes three neurons 164, and layer H₂ includes two neurons 166. Any number N of layers may be included in the neural network 160.

Associated with every connection (shown by lines connecting two (but could be more) neurons to each other) is a weight coefficient. Each hidden and output neuron sums its weighted inputs possibly along with an additive bias B. For example, an individual bias value B can be calculated for each neuron. The weights (W) and biases (B) are referred to as parameters, and each can be calculated using, e.g., training data and an iterative optimization algorithm as discussed above. Once the parameters are found, the whole network can then be used as a straightforward computation of inputs giving outputs.

When the network is used to calculate a prediction including a health score (e.g., an AKI score), which is computed every time a new input vector (X) is presented. A confidence interval or score may be presented with each health score.

An output of the prediction algorithm can be an AKI score (as described), a reversible kidney damage score, or an intrinsic or extrinsic kidney disease score, or a pre-renal, intra-renal, or post-renal injury score. Predicting kidney disease that is reversible or not, or intrinsic or extrinsic, helps to link the prediction output to an actionable (meaningful) therapeutic response.

The output module 108 receives results from the prediction algorithm, stores the data to an outputs/results database (OutDB) for future use in learning/updating of the prediction algorithm or retrieval, runs a set of display rules and rendering logic to provide instructions on what should be displayed and how it should be displayed on the user interface 110, and/or presents the kidney health results to the user interface 110 for display. In other embodiments, the display logic may run on the host computer where the user interface 110 is accessed.

Examples of outputs include (depending on the different embodiment and what it was trained to output) a kidney health score (e.g., an AKI stage, percentage of kidney function, severity of kidney damage, etc.), whether the predicted condition is an intrinsic or extrinsic kidney disease, whether the condition is a pre-renal, renal, or post-renal kidney disease, and/or whether the kidney condition is a reversible kidney injury, a time to kidney injury and/or other relevant information. Outputs may also include suggestions such as further diagnostic tests and/or therapy options (e.g., after assessing kidney health and processing other patient clinical and physiological information).

A prediction of a kidney condition that is output according to embodiments described herein may include a classification, description and/or other detail sufficient to allow a physician or other user to readily identify an appropriate therapy or treatment. For example, the prediction includes a description of a disease or condition that is known to have an associated therapy or treatment, a description of a disease or condition that is closely related to a known therapy or treatment, or at least includes a description that has sufficient detail and is specific enough to allow a user to identify an appropriate therapy or treatment. Examples of such detail include whether the predicted condition is an intrinsic or extrinsic kidney disease and/or whether the condition is a pre-renal, renal, or post-renal kidney disease.

Thus, in any of the aspects or embodiments described herein, the description provides methods of treating a disease or condition, e.g., AKI, comprising the steps of performing a method as described herein to predict, diagnose or characterize the disease or condition, and further including a step of administering a therapeutic modality, e.g., pharmacologic or procedural, or modifying an existing treatment regimen, wherein the treatment or modification of an existing treatment regimen is effective for treating or ameliorating a symptom of the disease or condition, e.g., AKI. In certain embodiments, the pharmacologic therapeutic comprises at least one of a steroid, cyclophosphamide, a diuretic such as furosemide, a vasopressor or a combination thereof. In certain embodiments, the therapeutic procedure comprises hemofiltration, hemodialysis, surgery, or the like. In certain embodiments, modifying an existing treatment regimen includes discontinuing the administration of a therapeutics, e.g., an ACE inhibitor, ARB antagonist, aminoglycoside, penicillin, NSAID or paracetamol.

The presentation of the results of the prediction algorithm can be in the form of, e.g., tabulated values, plots of current or historic values in time, with or without confidence intervals, inputs to subsequent inference algorithms that could be used for specificity of diagnosis or for therapy, other visualization means (e.g. damage % specified at the spatial (anatomical) or functional region), likelihood to be reversible, counter/timer until injury event, trajectory indicator of illness or forecast (e.g., where magnitude of score/damage is indicated by length or width/boldness of arrow, and direction indicates slope/trend from historic values or toward future values) and others. Results can be presented, e.g., in tabular form and/or in graphical form where desired on a static or a mobile monitor.

Various aspects of the system can be customized or configured by a user, for example, through the user interface 110. For example, the interface can be used to allow a user to select how inputs are calculated (e.g., choice of how to calculate base SCr, choice of how to calculate estimated GFR), and allow the user to select the models used in generating predictions. These user selections will change which formula for estimating inputs 153 is used in 150 and/or which model 155 is used in 150, and/or which computer algorithm is used in the prediction module 106 of 100.

Referring again to FIG. 2, various embodiments are discussed below in conjunction with a computer program, such as a program or application that includes one or more of the modules 102-118, and a graphical user interface such as the user interface 110, which are used to perform aspects of the above method(s). For example, the computer system 10 or components thereof can include a mobile application (“app”) that can be executed in a mobile device such as a smartphone, smart watch, tablet, etc. Also, it is noted that the particular format, configuration, naming, labels and other features of the computer program and display described below are provided for illustrative purposes and are not intended to limit the embodiments. The application can be network (Internet) based, e.g., hosted by a network, and/or the application can be run locally (e.g., downloaded onto a device).

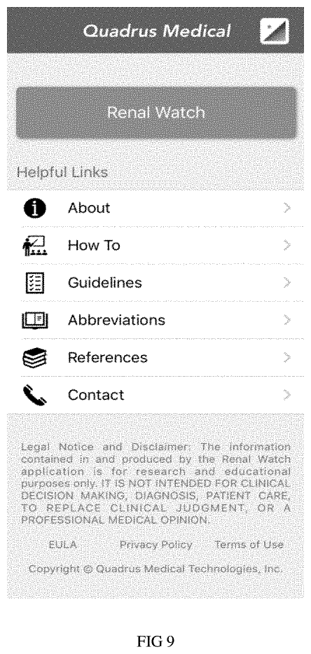

Referring to FIG. 9, on first launch of the application, the app would display a prompt screen that prompts a user to accept several agreements, including but not limited to terms of use, end user license agreement, and privacy policy. Upon subsequent application launch (and/or login), a user can be presented with a menu view. From the menu, one is presented with a product that includes software for performing renal evaluation and recommendation, which may be referred to as a “Renal Watch” product (calculator for AKI stage and severity). Other products may also be listed or otherwise presented. The app may display a button or other interface that allows a user to launch the application. For example, as shown in FIG. 9, the application can display a “Renal Watch” button that causes a processor to launch features of the application.

Additionally, as shown in FIG. 9, the user may be presented with a list of helpful links that may aid in understanding how to use one or more products or features. These links include, for example, “About,” “How To,” “Guidelines,” “Abbreviations,” “References”, and “Contact.” From each of the respective links, users can obtain information on what the product is about, how to use or interact with it, which guidelines (models, algorithms, rules, etc.) are being calculated or evaluated, which abbreviations are used in the app and what they mean, which references (publications, books, etc.) the user can refer to for more detailed information on the guidelines (models, algorithms, rules, etc.). Lastly, the user can see contact information for the company or provider of the product, and can include additional information, demos, or product support.

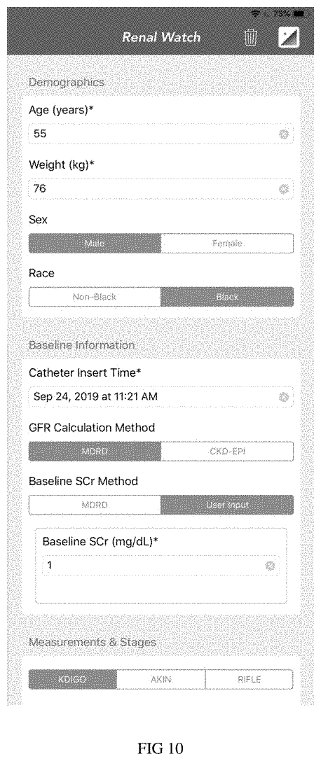

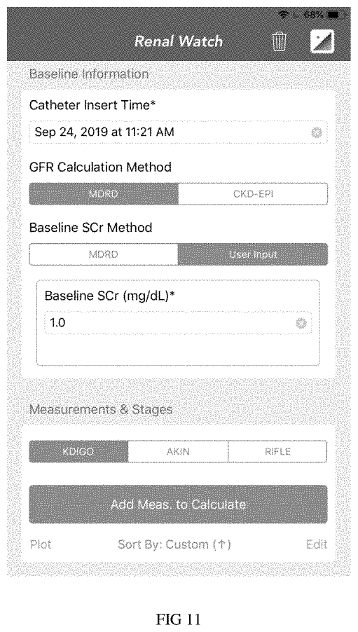

Upon clicking the Renal Watch button in FIG. 9, the user is directed to a product view (FIG. 10). The user is then (or instead) directed to an input display that includes fields for entering demographics and baseline information. For example, the display of FIG. 10 includes a “Demographics” section, a “Baseline Information” section. The display may also include a “Measurements and Stages” section for entry of measurements taken for a patient, as shown in FIG. 10 and FIG. 11.

The user enters the demographic information (e.g., age, weight, sex, and race) and baseline information (catheter insert or first UO (urine output) measurement time), a GFR (glomerular filtration rate) calculation method, a baseline SCr (serum creatinine) calculation method, and/or baseline SCr information. The fields labeled with an asterisk require a user selection; those without it can use default or already/previously selected values. For example, the default or already/previously selected sex is male and the default or already/previously selected GFR method is Modified Diet in Renal Disease (MDRD). Other embodiments may include additional selections for calculating a baseline serum creatinine or estimated GFR, such as the Chronic Kidney Disease-Epidemiology Collaboration and the Cockcroft Gault formulas. Clicking the encircled x to the right of an input/edit box will clear its contents and allow for re-entry.

It is noted that the manual data entry in the embodiments described herein can also occur automatically via the aforementioned input module 102 (FIG. 2) or data acquisition or data retrieval system or can occur both automatically and manually.

FIGS. 9-32 depict examples of various displays and user interfaces that may be presented to a user by the Renal Watch application discussed above. Aspects of the Renal Watch application may be presented as various sections in the formats shown, or otherwise presented in any suitable format. In addition, although the displays and interfaces are shown in Mobile device displays, they are not so limited and may be presented or displayed using any suitable device or system, such as a personal computer, desktop computer etc.

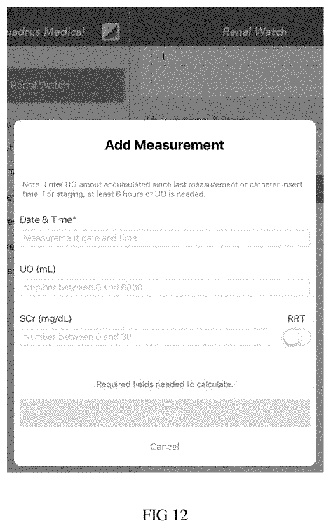

FIG. 11 shows a “Measurements & Stages” section, by which a user can select a guideline to be used to calculate a patient's stage or severity of kidney injury (0=absent, 1=mild, 2=moderate, 3=severe). Guidelines that can be selected include, for example, KDIGO (Kidney Disease Improving Global Outcomes), AKIN (Acute Kidney Injury Network), RIFLE (Risk Injury Failure Loss and End Stage) criteria, and/or other criteria. From this view, the user also has the option to plot data and/or results, sort the data and/or results, and edit the data. The user can add other measurements by engaging (e.g., clicking or tapping) an “Add Meas. To Calculate” button, which launches an “Add Measurements View” as shown in FIG. 12. In some embodiments, this button could be an encircled ‘+’ icon or could read “+Add Measurement”.

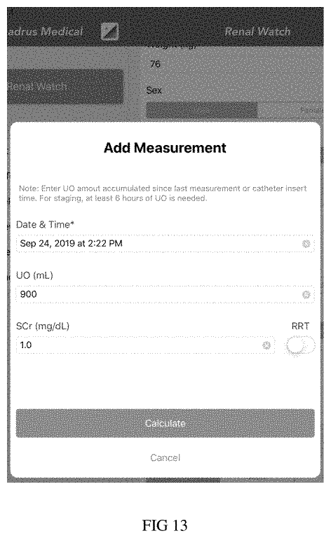

Referring to FIG. 13, to add a measurement, the user can enter the date and time, as well as additional information related to previous measurements or status. For example, the UO accumulated since last measurement, the serum creatinine, and/or the RRT (renal replacement therapy) status (e.g. patient is on/receiving dialysis) can be entered. In another example, the urine output accumulation and the time over which the UO accumulated can both be entered. In some embodiments, units of measure can be input by manual input by the user or selection from a drop-down menu, so that the user may enter measurement values in their preferred measurement units and the Renal Watch application would do any required conversion.

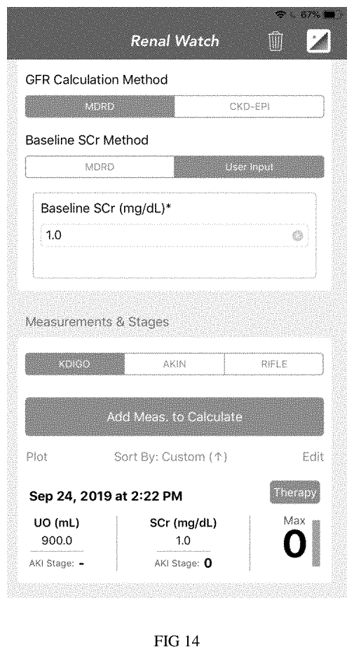

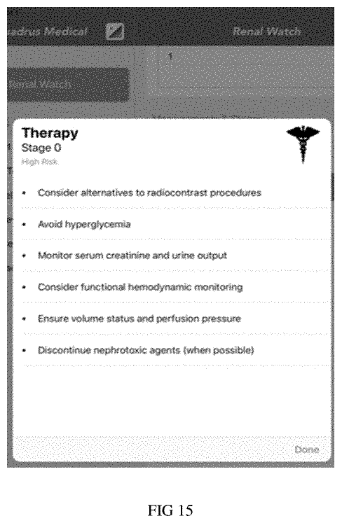

Once data entry is entered in the Add Measurement modal (FIGS. 12 and 13), a user clicks the Calculate button to calculate the AKI stage and view the result. FIG. 14 shows the Measurements & Stages section after the calculation is performed, which shows the result of the calculation. In this example, the result is shown as including the new measurement data and the calculated result AKI Stage.

In the example of FIG. 14, items are displayed from left to right include the new UO measurement (900 mL) and its corresponding stage (-) meaning not applicable or not able to be calculated (e.g. insufficient data entry or time period). The displayed items also include the new SCr measurement (1.0 mg/dL) and its corresponding AKI stage (zero), and the maximum (“Max”) AKI Stage. The Max AKI Stage is the greater of the AKI Stage by UO and the AKI Stage by SCr criteria, which in this example is zero. The Max AKI stage can be represented in a graphical format. For example, a vertical color bar or other graphic indicates the stage visually (stage 0=green, stage 1=yellow, stage 2=orange, stage 3=red). Other aggregators or operators can be used for selection of the overall/max stage.

The user can tap or otherwise engage a “Therapy” button to prompt the Renal Watch application to present a therapy recommendation. It is noted that references to tapping or clicking are examples of how a user can interact with displayed features, and are not intended to limit how a user can engage with a feature to prompt a certain function.

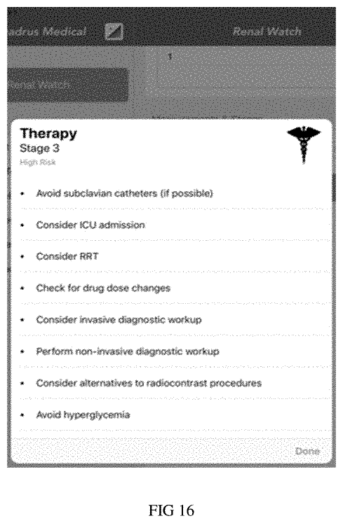

Referring to FIG. 15, upon clicking the Therapy button, a Therapy modal or screen shows the therapy recommendations corresponding to the overall/max stage. Depending on the patient's stage and risk profile, the recommendations may change. In this example, the Therapy screen presents a number of therapy recommendations for a stage zero patient. FIG. 16 shows an example of displayed therapy recommendations for a stage 3 patient. The recommendations are presented in FIGS. 15 and 16 as a bulleted list, but can also be shown as, for example, checkboxes or radio buttons that upon clicking can show an indication of acknowledgment or completion (e.g. strikethrough text as on a checklist).

If no recommendations are available, the Renal Watch application can prompt a user to enter information and complete a high-risk checklist assessment for the patient's chronic or acute conditions or extrinsic exposures (e.g., community, environmental, infection, etc.). Upon determining the high-risk assessment, the recommendations can be updated.

In further embodiments, each therapy recommendation can be clicked or otherwise engaged to open a new screen revealing relevant data needed to support a user's action for compliance with the recommendation. For example, clicking the recommendation “Check for drug dose changes” (FIG. 16) can open a new screen with all administered and/or ordered/prescribed medications, highlighting those that have recently changed dose and/or highlighting those that are considered to be nephrotoxic. In further embodiments, recommendations for alternative drugs and/or doses could be provided.

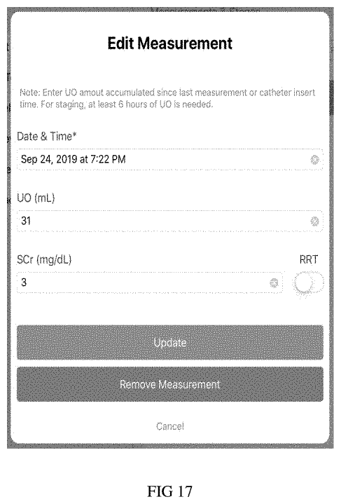

A user can edit or adjust measurement data by engaging, for example, the “Measurements & Stages” section by tapping/clicking of a displayed measurement. The user will then be shown an “Edit Measurement” screen, an example of which is shown in FIG. 17. Here the user can adjust the measurement data if an error was made or if updated information becomes available. Clicking “Update” will save the change and return the user to the “Measurements & Stages” view, while clicking “Cancel” will not save changes and will return the user to the “Measurements & Stages” view. Clicking “Remove Measurement” will delete the measurement (or start a process of deleting the measurement). The user can optionally be prompted with a confirmation screen (e.g. “Are you sure you want to delete this measurement?”), from which they can confirm deletion of the measurement or cancel to return to the “Edit Measurement” screen.

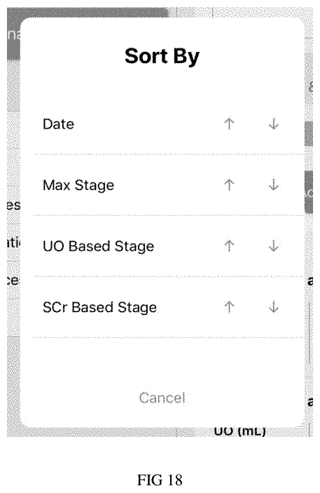

Upon tapping/clicking of a “Sort By” button in the “Measurements & Stages” section, a sort modal displays data attributes which can be used to sort the data in ascending or descending order by clicking the respective arrows (e.g. Date descending or Max stage ascending). An example of the data attributes is shown in FIG. 18. Clicking “Cancel” will undo will return the user to the “Measurements & Stages” view. Custom sorting occurs either by user-entry (the order in which the user added the measurements) or by clicking “Edit” and shuffling the rows manually by click-and-drag functionality.

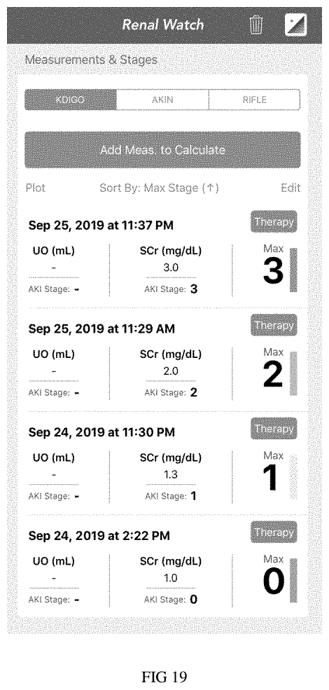

FIG. 19 shows an example of sorted measurement data, which has been sorted according to the selected data attributes. In this example, various measurements are sorted by associated PKI stage, but can be sorted in other ways as well.

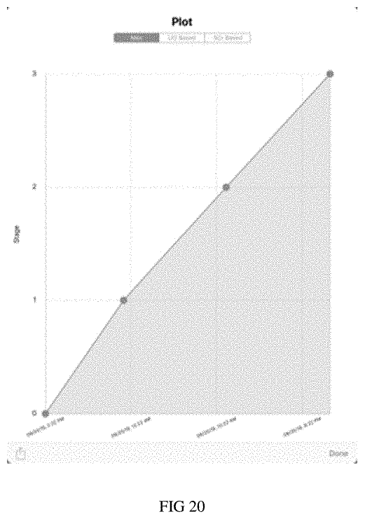

The user can view the measurement and/or stage data in formats other than a list. For example, the data can be plotted or otherwise displayed in a graphical format. In FIG. 19, a user can plot data by clicking “Plot” in the Measurements and Stage Plot view. The plotted data can be displayed in a “Plot” section shown in FIG. 20. The measurement data can be plotted as a function of AKI stages and/or as a function of time. The user can select to plot the AKI stage by max operator (or other aggregator), by UO criteria, or by SCr criteria. The user can click “Done” to close the plot and return to the “Measurements & Stages” view. In other embodiments, the UO and/or SCr inputs (and their respective decision thresholds or cutoffs for deciding stage 1, 2, or 3) can also be plotted in time. In other embodiments, the plot view could plot AKI staging for one or more guidelines (KDIGO, AKIN, RIFLE) and allow comparisons of AKI stage by the one or more guidelines (KDIGO, AKIN, RIFLE), or of the AKI stage of the one or more guidelines the under different initial conditions (e.g., baseline serum creatinine, estimated glomerular filtration rate, etc.).

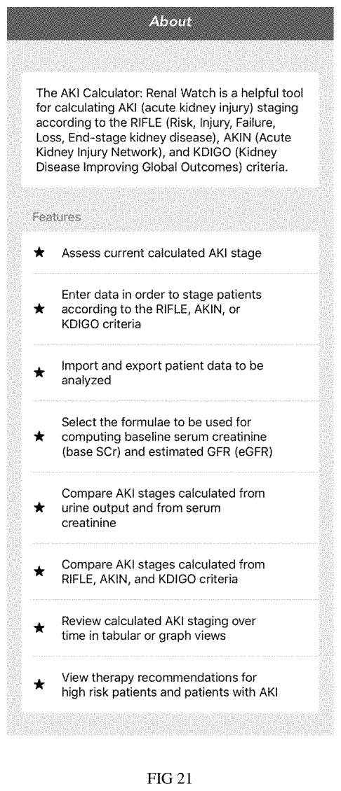

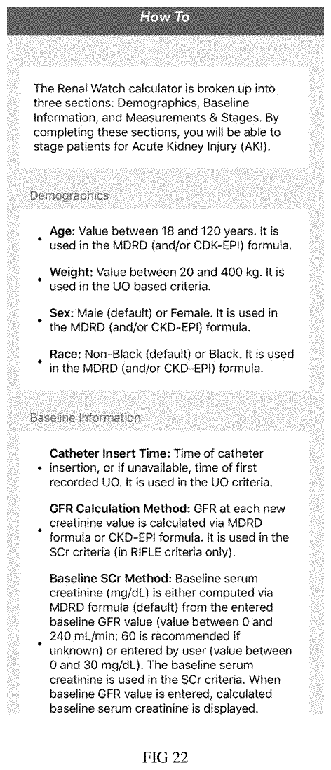

FIG. 21-32 show examples of displays prompted by a user selecting links shown in FIG. 9. FIG. 21 shows an example of an “About” screen displayed by selecting the “About” link. FIG. 22 shows an example of a screen displayed in response to a user selecting the “How To” link. When these links are selected from the menu bar of FIG. 9, or a navigation bar or hamburger menu, kebab menu, or similar menu icon, the user will be able to read about the product and how to use it, including descriptions of each section, default values, ranges of valid values, etc.

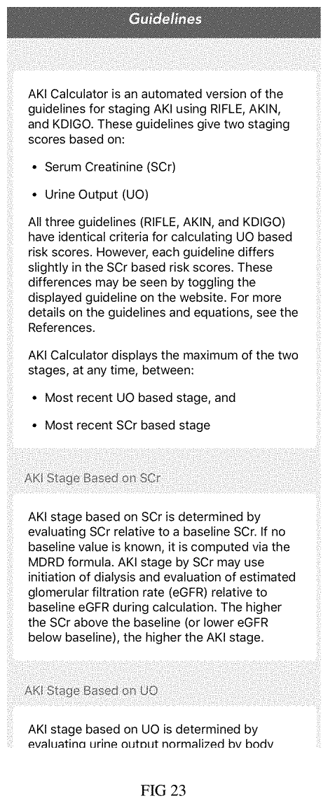

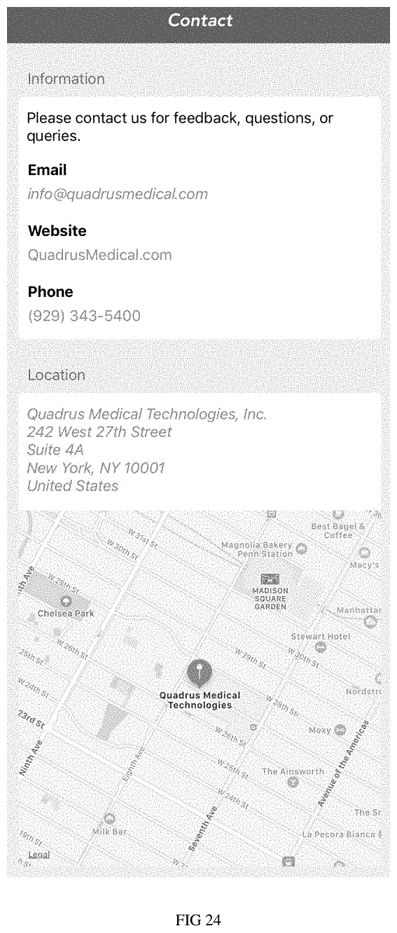

Examples of “Guidelines” and “Contact” views are shown in FIG. 23 and FIG. 24, respectively. When these items are selected, the user will be able to read about the guidelines being used to provide kidney health stage and therapy recommendations and find contact information for the company, technical support, etc. In some embodiments, the guidelines view could show information on how the forecasted or predicted kidney health was calculated.

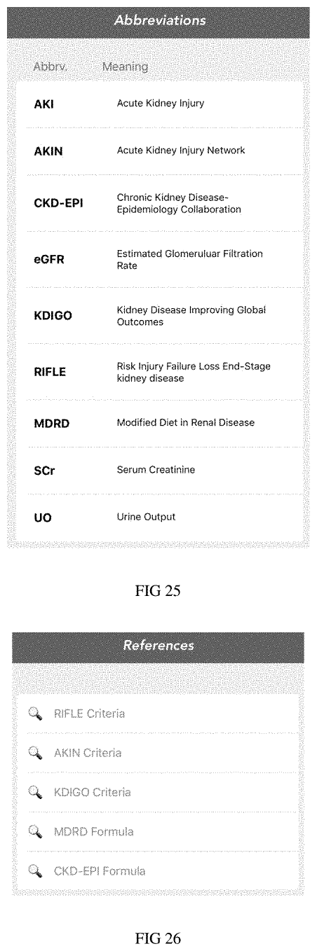

An “Abbreviations” section (FIG. 25) display commonly used abbreviations and acronyms that appear in the application content. The “References” section (FIG. 26) includes links to source or reference material for the respective guidelines or formulae, e.g. where we obtain the rules that we implemented if not generated from our own development. Other available models could also be listed there with the reference linked to relevant journal or conference publications or user manuals.

The “References” section may include links to source or reference material for the respective guidelines, formulas, and/or therapy recommendations. These may be links to internet pages, scientific articles, publications, etc. containing information about the rules implemented, their performance, or other pertinent information.

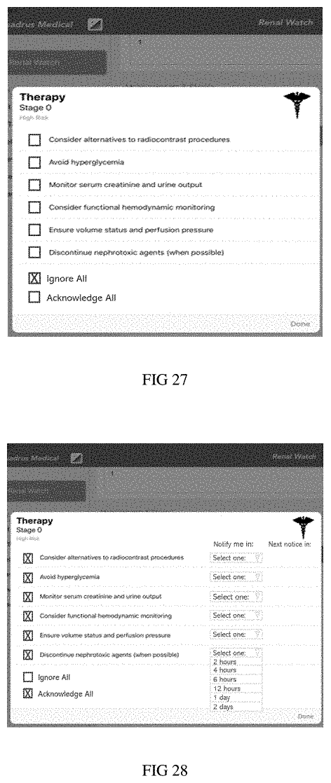

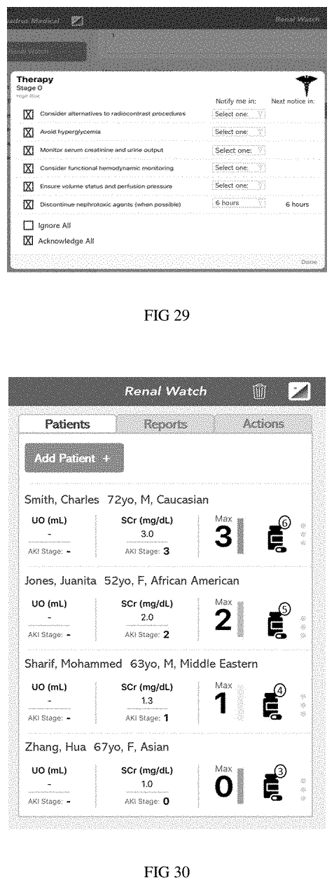

Additional examples of the “Therapy” screen are shown in FIGS. 27-29. These examples illustrate other therapy options, such as a status (ignore or acknowledge) and a notification feature (when to provide a reminder or re-notify the user of the guidelines for acknowledgement). In the example of FIG. 27, “Ignore All” is selected, so that none of the checkboxes are selected, whereas in FIG. 28, “Acknowledge All” is selected, so that all of the checkboxes are selected. While checkboxes are shown, they can be radio buttons, sliders or others. FIGS. 28 and 29 show examples of notification features that include a selection of a time to be notified and a display of the next reminder time (for those already scheduled or selected). Similarly, the notification time can be displayed/selected from one or more drop-down menus, or scroll wheels, with numbers (e.g. 2, 4, 6) and units (e.g., minutes, hours, days) displayed/selected separately or together. If a notification for one of the recommendations was already scheduled, the time until next notice would be shown.

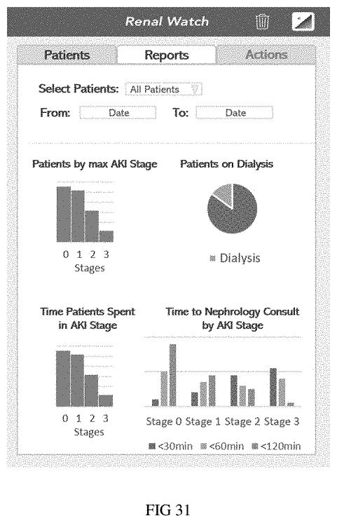

FIG. 30 depicts a user profile view, population view, or multi-patient view. This allows a user (e.g. physician, nurse, etc.) to view of all current patients that he/she is treating, their demographics, baseline health information, most recent measurement(s), current AKI stages, and most recent therapy recommendations, and the actions/notifications pending. The actions/notifications pending can be indicated, for example, an encircled number above corresponding pill bottle illustrations. Clicking the kebab menu (three vertical dots) of an individual patient would allow you to edit the patient information or discharge the patient. Other embodiments can also include the predicted AKI stage and the predicted trend in AKI stage. Clicking “Add Patient” would open a screen prompting the user for entering information about a patient, such as the demographics, and baseline health information shown in.

As shown in the exemplary reports view of FIG. 31, users can generate reports on all patients (e.g., current/admitted and historic/past or previously discharged patients), all patients treated during a certain time period, all current patients (currently admitted or being treated), or historic patients (those previously treated and now discharged/deceased). The reports view allows a user (e.g. Intensive Care Unit (ICU) physician or ICU director) to assess how a care area or unit is doing with respect to quality outcomes of interest. It can also be used by a user (e.g. hospitalist or other responsible for resource allocation) to determine number of nurses or dialysis machines required, review ICU stats, etc. based on details of the patient population, their kidney health, or the types of therapies they are receiving.

In other embodiments, a reports view may also be available for an individual patient. In such a view, trend information on the patient's health progression can be displayed, as well as amount of fluids, meds, or intervention, or timing of those interventions, relative to disease (stage) onset or progression.

In other embodiments, a scenarios button and view could enable the user to run scenarios of different guidelines, baselines, initial conditions, or assumptions and show in plot, tabularized, or summary/report view the resulting current or forecasted kidney health stage under these scenarios. This could be presented with a confidence interval over all scenarios run. In other embodiments, the scenarios can be intervention scenarios and the resulting forecasting kidney health under different interventions can be shown. Further, the therapy recommendations can include can be customized per geographical region or can be enhanced to show those that are most cost effective. For instance, suggested or recommended drugs can be displayed with their approximate cost in a particular geographical region.

In other embodiments, the AKI stage can be the presence or severity of other kidney conditions or diseases and the forecasting of those kidney conditions in time.

In other embodiments, a multi-patient view can be included that displays the predicted AKI stage and other predicted renal health information. The therapy recommendations and/or the actions/notifications may be updated to reflect additional prophylactic or preventive interventions. An additional icon may be used. Trend information or a plot of forecasted renal health or disease stage may be shown on this screen.

The “Actions” tab of the profile view (FIG. 31) contains a summary view of all of the actions for the end user across all of the patients s/he is responsible for treating.

The menu bar or main screen where the user logs in may also contain several navigation options and ways to change or update profile, preferences, and ways to change or upgrade product or license subscription. If the application main screen eventually provides a portal to multiple products, the user can be shown a list of possible products upon login and would select the application s/he wishes to run. Alternatively, a switch can be applied (e.g. slider or drop down) to allow the provider to switch from a renal health focused application to a lung or heart focused application.

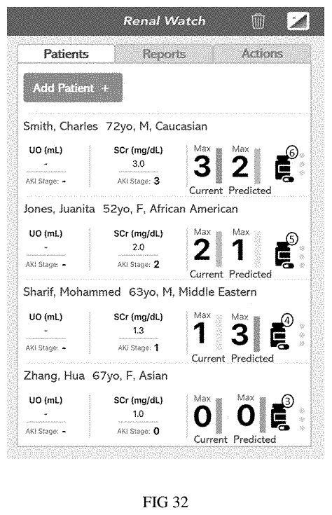

In other embodiments, when forecasted renal stage is displayed, the main page may include a learning or prediction button. Upon clicking, the learning button on the main page provides an option where a user decides to re-train the prediction algorithm based on the patients they have seen in a pre-determined or customizable number of days or weeks. The prediction button would update the prediction of kidney health (including a predicted stage) a pre-determined or customizable number of hours or days in the future. FIG. 32 shows one such way that a predicted or forecasted stage can be shown to the user.

### Characteristics

The following is a list of characteristics and features of the embodiments described herein. It is noted that all of the characteristics or features may be included, or a subset thereof may be included.
Application enabling reviewing and analyzing current or predicted kidney health stage and therapy recommendations for one or more patients. This feature can be used for creating, updating, or modifying diagnostic or therapeutic plans for a patient's kidney health
Application comprising a profile (multi-patient) view and an individual patient view. The profile view enables a user to view, e.g., demographics, baseline health information, current or forecasted kidney health stage/score, therapy recommendation that a user is treating or all patients in a given care unit of a hospital or health facility. The profile view can include an individual view for showing kidney health stage and therapy recommendation
Profile view with reports view enabling a user to select current or historic (previously discharged) patients, view summary information, and/or generate reports of all patients that a user is treating or all patients in a given care unit of a hospital or health facility. This can be used for quality improvement studies or performance indicators, resource (machine, equipment, staff) allocation, etc.
Therapy recommendation view with features for displaying, acknowledging/ignoring, requesting notifications, and following up on previously scheduled notifications.
Therapy recommendation view whereby specific recommendations can be clicked to drill down to display data needed to act upon that recommendation. Therapy recommendation may also include cost or cost-effectiveness and can be customized per region
On demand learn button to update the guidelines or update (retrain) the model.
On demand predict button to update the forecasted or predicted kidney stage, score, or probability.
On demand scenarios button to compute the kidney health stage under different initial conditions (baselines, formulas, guidelines, or assumptions).
On demand intervention scenarios button to compute the kidney health stage under different interventions.
User preferences for baseline health information and guidelines that can be customized and stored for one or more patient, including selection of the formulas used to compute baseline serum creatinine, estimated glomerular filtration rate, or kidney health stage or to provide kidney health recommendation.
Plotting view that allows comparison of kidney health stages by one or more inputs, by one or more guidelines, by one or more initial conditions (scenarios), by one or more formulas (scenarios), by one or more interventions (scenarios), etc.
High-risk checklist to select patient chronic or acute health conditions or extrinsic (community) exposures and update therapy recommendations accordingly.
Electronically captured or auto-charted data via a data acquisition or retrieval system that sends and receives messages to establish communication with medical devices and systems, receives information or messages containing information and/or data, parses or processes the data according to standard or proprietary protocols, stores the data in a file and/or repository accessible by the application; alternatively and/or additionally, it sends the data to the application. Communication can be wired or wireless.

The following are embodiments of the present invention:

### Embodiment 1

A system for assessing kidney health, the system comprising: a processing device including: an input module configured to receive input values related to kidney function of a patient; a prediction module comprising a computation algorithm and/or a model configured to predict a kidney condition and calculate a kidney health score related to at least one of a severity and a probability of the predicted kidney condition, the kidney health score calculated based on the one or more input values; and an output module configured to present the predicted kidney condition and the kidney health score to a medical professional.

### Embodiment 2

The system of one or more embodiments, wherein the output module is configured to perform at least one of: presenting a diagnostic protocol for diagnosing the predicted kidney condition, and recommending one or more diagnostic tests for evaluating the kidney function.

### Embodiment 3

The system of one or more embodiments, wherein the output module is configured to present at least one of a treatment protocol for treating the predicted kidney condition, and a recommendation as to an adjustment of an existing treatment protocol.

### Embodiment 4

The system of one or more embodiments, wherein the output module is configured to store the predicted kidney condition and the kidney health score, and output at least one of a textual, audial, and visual representation of the predicted kidney condition in at least one of an e-mail, an SMS message, an alert, an alarm, a graphical user interface and a display.

### Embodiment 5

The system of one or more embodiments, wherein at least one of the prediction module, the computation algorithm and/or the model is configured to calculate at least one of a level of confidence and a probability that the predicted kidney condition and the kidney health score are accurate.

### Embodiment 6

The system of one or more embodiments, wherein the input values include at least one known input value and/or at least one estimated input value, and the at least one of the level of confidence and the probability is calculated based on a combination of the input values and performance of the model and/or the algorithm, the model and/or the algorithm configured to output the kidney health score based on the input values.

### Embodiment 7

The system of one or more embodiments, wherein the at least one known input value is at least one of a measured physiological variable, a vital sign, a lab test result, a demographic, a comorbid condition, and an intervention (e.g. dialysis, fluid, or medication).

### Embodiment 8

The system of one or more embodiments, wherein the at least one estimated input value is estimated using at least one of an inference, a correlation, a regression, an algebraic equation, an ordinary differential equation and a partial differential equation.

### Embodiment 9

The system of one or more embodiments, wherein the prediction module is configured to calculate a probability that the predicted kidney condition is accurate, and calculate the level of confidence based on the probability.

### Embodiment 10

The system of one or more embodiments, wherein the probability includes at least one of a probability score and a probability distribution.

### Embodiment 11

The system of one or more embodiments, wherein the probability score is calculated by performing at least one of: predicting the kidney health score according to a first guideline, rule or model and generating a first prediction, predicting the kidney health score according to a second guideline, rule or model and generating a second prediction, comparing the first prediction to the second prediction, and estimating a probability that the patient has the predicted kidney condition based on the comparison; and randomly selecting a first plurality of input values and performing a first prediction of the kidney health score according to the first guideline, rule or model, randomly selecting a second plurality of input values and performing a second prediction of the kidney health score according to the first guideline, rule or model, comparing the first prediction to the second prediction, and estimating a probability that the patient has the predicted kidney health score based on the comparison.

### Embodiment 12

The system of one or more embodiments, wherein the random selection is based on a Monte Carlo-like simulation or bootstrapping or similar approach or simulation on perturbations of at least one of the input values, a guideline, a rule, a model used for estimating outputs, and a formula or a model used for estimating inputs.

### Embodiment 13

The system of one or more embodiments, further comprising a pre-processing module configured to pre-process the input values and store the pre-processed input values and processed health data to an inputs database.

### Embodiment 14

The system of one or more embodiments, wherein the pre-processing module is configured to train a learning algorithm based on health data from a plurality of patients, the health data including data related to healthy patients and data related to patients having the predicted kidney condition.

### Embodiment 15

The system of one or more embodiments, wherein the kidney health score is calculated based on a trained computation/prediction algorithm, the training performed by a learning algorithm, the learning algorithm trained based on health data from a plurality of patients, the health data including data related to healthy patients and data related to patients having the predicted kidney condition.

### Embodiment 16

The system of one or more embodiments, wherein the learning algorithm includes a mathematical process to update weights, coefficients, biases, and/or parameters of a nonlinear mapping function of the input values to one or more output values, the one or more output values including the kidney health score.

### Embodiment 17

The system of one or more embodiments, wherein the nonlinear mapping function includes a set of rules or guidelines.

### Embodiment 18

The system of one or more embodiments, wherein the learning algorithm includes a deep learning neural network (DLNN).

### Embodiment 19

The system of one or more embodiments, wherein the learning algorithm includes a differential equation-based model where parameters have physiological meaning, or a differential equation-based model where parameters do not have physiological meaning (e.g. time series models).

### Embodiment 20

The system of one or more embodiments, wherein the prediction module is configured to update the trained computation/prediction algorithm based on new health data from a plurality of patients.

### Embodiment 21

The system of one or more embodiments, wherein the plurality of patients are selected over a selected range of time from at least one of a selected care unit or facility, a selected geographical location, and a selected subset of a patient population.

### Embodiment 22

The system of one or more embodiments, wherein the prediction module is configured to calculate the kidney health score based on a nonlinear mapping function of the input values to one or more output values, the one or more output values including the kidney health score.

### Embodiment 23

The system of one or more embodiments, wherein the nonlinear mapping function is a deep learning neural network (DLNN).

### Embodiment 24

The system of one or more embodiments, wherein the nonlinear mapping function is a differential equation-based model where parameters have physiological meaning, or a differential equation-based model where parameters do not have physiological meaning (e.g. time series models).

### Embodiment 25

The system of one or more embodiments, further comprising a post-processing module configured to perform at least one of: storing the predicted kidney condition and the kidney health score in a results database, and performing an inference using the kidney health score to provide an advisory to a user.

### Embodiment 26

The system of one or more embodiments, wherein the advisory is at least one of a diagnostic protocol, a therapeutic protocol and an adjustment to a therapy.

### Embodiment 27

The system of one or more embodiments, wherein the input module is configured to establish communication with a medical device and/or system and receive input data therefrom.

### Embodiment 28

The system of one or more embodiments, wherein the predicted kidney condition is at least one of an acute kidney injury (AKI), reversible kidney damage, an intrinsic kidney disease, an extrinsic kidney disease, a pre-renal condition, an intrarenal condition, and a post-renal condition.

### Embodiment 29

The system of one or more embodiments, wherein the predicted kidney condition is represented by at least one of a predicted AKI stage, a percentage of remaining kidney function and a percentage of a kidney that is injured or damaged.

### Embodiment 30

A method of assessing kidney health, the method comprising: receiving, by an input module, input values related to kidney function of a patient; predicting, by a prediction module comprising a computation algorithm and/or a model, a kidney condition and calculating a kidney health score related to at least one of a severity and a probability of the predicted kidney condition by a prediction module, the kidney health score calculated based on the one or more input values; and presenting, by an output module, the predicted kidney condition and the kidney health score to a medical professional.

### Embodiment 31

The method of one or more embodiments, further comprising performing, by the output module, at least one of: presenting a diagnostic protocol for diagnosing the predicted kidney condition, and recommending one or more diagnostic tests for evaluating the kidney function.

### Embodiment 32

The method of one or more embodiments, further comprising presenting, by the output module, at least one of a treatment protocol for treating the predicted kidney condition, and a recommendation as to an adjustment of an existing treatment protocol.

### Embodiment 33

The method of one or more embodiments, further comprising storing the predicted kidney condition and the kidney health score, and outputting at least one of a textual, audial, and visual representation of the predicted kidney condition in at least one of an e-mail, an SMS message, an alert, an alarm, a graphical user interface and a display.

### Embodiment 34

The method of one or more embodiments, further comprising calculating at least one of a level of confidence and a probability that the predicted kidney condition and the kidney health score are accurate.

### Embodiment 35

The method of one or more embodiments, wherein the input values include at least one known input value and at least one estimated input value, and the at least one of the level of confidence and the probability is calculated based on a combination of the input values and performance of the algorithm and/or the model, the algorithm and/or the model configured to output the kidney health score based on the input values.

### Embodiment 36

The method of one or more embodiments, wherein the at least one known input value is at least one of a measured physiological variable, a vital sign, a lab test result, a demographic, a comorbid condition, and an intervention (e.g. dialysis, fluid, or medication).

### Embodiment 37

The method of one or more embodiments, wherein the at least one estimated input value is estimated using at least one of an inference, a correlation, a regression, an algebraic equation, an ordinary differential equation and a partial differential equation.

### Embodiment 38

The method of one or more embodiments, wherein the prediction module is configured to calculate a probability that the predicted kidney condition is accurate, and calculate the level of confidence based on the probability.

### Embodiment 39

The method of one or more embodiments, wherein the probability includes at least one of a probability score and a probability distribution.

### Embodiment 40

The method of one or more embodiments, wherein the probability score is calculated by performing at least one of: predicting the kidney health score according to a first guideline, rule or model and generating a first prediction, predicting the kidney health score according to a second guideline, rule or model and generating a second prediction, comparing the first prediction to the second prediction, and estimating a probability that the patient has the predicted kidney condition based on the comparison; and randomly selecting a first plurality of input values and performing a first prediction of the kidney health score according to the first guideline, rule or model, randomly selecting a second plurality of input values and performing a second prediction of the kidney health score according to the first guideline, rule or model, comparing the first prediction to the second prediction, and estimating a probability that the patient has the predicted kidney health score based on the comparison.

### Embodiment 41

The method of one or more embodiments, wherein the random selection is based on a Monte Carlo-like simulation or bootstrapping or similar approach or simulation on perturbations of at least one of the input values, a guideline, a rule, a model used for estimating outputs, and a formula or a model used for estimating inputs.

### Embodiment 42

The method of one or more embodiments, further comprising pre-processing the input values and storing the pre-processed input values and processed health data to an inputs database by a pre-processing module, wherein the pre-processing includes at least one of filtering, outlier removal, and scaling or normalizing.

### Embodiment 43

The method of one or more embodiments, wherein the pre-processing includes training a learning algorithm based on health data from a plurality of patients, the health data including data related to healthy patients and data related to patients having the predicted kidney condition.

### Embodiment 44

The method of one or more embodiments, wherein the kidney health score is calculated based on a trained computation/prediction algorithm, the training performed by a learning algorithm, the learning algorithm trained based on health data from a plurality of patients, the health data including data related to healthy patients and data related to patients having the predicted kidney condition.

### Embodiment 45

The method of one or more embodiments, wherein the learning algorithm includes a mathematical process to update weights, coefficients, biases, and/or parameters of a nonlinear mapping function of the input values to one or more output values, the one or more output values including the kidney health score.

### Embodiment 46

The method of one or more embodiments, wherein the nonlinear mapping function includes a set of rules or guidelines.

### Embodiment 47

The method of one or more embodiments, wherein the learning algorithm includes a deep learning neural network (DLNN).

### Embodiment 48

The method of one or more embodiments, wherein the learning algorithm includes a differential equation-based model where parameters have physiological meaning, or a differential equation-based model where parameters do not have physiological meaning.

### Embodiment 49

The method of one or more embodiments, further comprising updating the trained computation/prediction algorithm based on new health data from a plurality of patients.

### Embodiment 50

The method of one or more embodiments, wherein the plurality of patients are selected over a selected range of time from at least one of a selected care unit or facility, a selected geographical location, and a selected subset of a patient population.

### Embodiment 51

The method of one or more embodiments, wherein the prediction module calculates the kidney health score based on a nonlinear mapping function of the input values to one or more output values, the one or more output values including the kidney health score.

### Embodiment 52

The method of one or more embodiments, wherein the nonlinear mapping function is a deep learning neural network (DLNN).

### Embodiment 53

The method of one or more embodiments, wherein the nonlinear mapping function is a differential equation-based model where parameters have physiological meaning, or a differential equation-based model where parameters do not have physiological meaning.

### Embodiment 54

The method of one or more embodiments, further comprising performing, by a post-processing module, at least one of: storing the predicted kidney condition and the kidney health score in a results database, and performing an inference using the kidney health score to provide an advisory to a user.

### Embodiment 55

The method of one or more embodiments, wherein the advisory is at least one of a diagnostic protocol, a therapeutic protocol and an adjustment to a therapy.

### Embodiment 56

The method of one or more embodiments, wherein the input module is configured to establish communication with a medical device and/or system and receive input data therefrom.

### Embodiment 57

The method of one or more embodiments, wherein the predicted kidney condition is at least one of an acute kidney injury (AKI), reversible kidney damage, an intrinsic kidney disease, an extrinsic kidney disease, a pre-renal condition, an intrarenal condition, and a post-renal condition.

### Embodiment 58

The method of one or more embodiments, wherein the predicted kidney condition is represented by at least one of a predicted AKI stage, a percentage of remaining kidney function and a percentage of a kidney that is injured or damaged.

### Embodiment 59

The method of one or more embodiments, further comprising the step of administering a therapeutic modality or modifying an existing treatment based on the output values, wherein the method effectuates the treatment or amelioration of at least on symptom of the predicted kidney condition.

The descriptions of the various embodiments of the present invention have been presented for purposes of illustration, but are not intended to be exhaustive or limited to the embodiments described. Many modifications and variations will be apparent to those of ordinary skill in the art without departing from the scope and spirit of the described embodiments. The terminology used herein was chosen to best explain the principles of the embodiments of the invention, the practical application or technical improvement over technologies found in the marketplace, or to enable others of ordinary skill in the art to understand the embodiments described herein.

What is claimed is:

1. A system for assessing kidney health, the system comprising:
a processing device including:
an input module configured to receive input values related to kidney function of a patient;
a prediction module comprising a computation algorithm and/or a model configured to predict a kidney condition and calculate a kidney health score related to at least one of a severity and a probability of the predicted kidney condition, the kidney health score calculated based on the one or more input values; and
an output module configured to present the predicted kidney condition and the kidney health score to a medical professional.

2. The system of claim 1, wherein the output module is configured to perform at least one of: presenting a diagnostic protocol for diagnosing the predicted kidney condition, and recommending one or more diagnostic tests for evaluating the kidney function.

3. The system of claim 1, wherein the output module is configured to present at least one of a treatment protocol for treating the predicted kidney condition, and a recommendation as to an adjustment of an existing treatment protocol.

4. The system of claim 1, wherein the output module is configured to store the predicted kidney condition and the kidney health score, and output at least one of a textual, audial, and visual representation of the predicted kidney condition in at least one of an e-mail, an SMS message, an alert, an alarm, a graphical user interface and a display.

5. The system of claim 1, wherein at least one of the prediction module, the computation algorithm and/or the model is configured to calculate at least one of a level of confidence and a probability that the predicted kidney condition and the kidney health score are accurate.

6. The system of claim 5, wherein the input values include at least one known input value and/or at least one estimated input value, and the at least one of the level of confidence and the probability is calculated based on a combination of the at least one known input value and/or the at least one estimated input value and performance of the model and/or the algorithm, the model and/or the algorithm configured to output the kidney health score based on the input values.

7. The system of claim 6, wherein the at least one known input value is at least one of a measured physiological variable, a vital sign, a lab test result, a demographic, a comorbid condition, and an intervention (e.g. dialysis, fluid, or medication), and the at least one estimated input value is estimated using at least one of an inference, a correlation, a regression, an algebraic equation, an ordinary differential equation and a partial differential equation.

8. The system of claim 5, wherein the at least one of the level of confidence and the probability is calculated by performing at least one of:
predicting the kidney health score according to a first guideline, rule or model and generating a first prediction, predicting the kidney health score according to a second guideline, rule or model and generating a second prediction, comparing the first prediction to the second prediction, and estimating a probability that the patient has the predicted kidney condition based on the comparison; and
randomly selecting a first plurality of input values and performing a first prediction of the kidney health score according to the first guideline, rule or model, randomly selecting a second plurality of input values and performing a second prediction of the kidney health score according to the first guideline, rule or model, comparing the first prediction to the second prediction, and estimating a probability that the patient has the predicted kidney health score based on the comparison.

9. The system of claim 8, wherein the random selection is based on a Monte Carlo-like simulation or bootstrapping or similar approach or simulation on perturbations of at least one of the input values, a guideline, a rule, a model used for estimating outputs, and a formula or a model used for estimating inputs.

10. The system of claim 1, wherein the prediction module is configured to calculate the kidney health score based on a nonlinear mapping function of the input values to one or more output values, the one or more output values including the kidney health score.

11. The system of claim 10, wherein the nonlinear mapping function is at least one of a multi-layer neural network (MLNN), deep learning neural network (DLNN), or recurrent neural network (RNN); or a set of guidelines or rules; or a differential equation-based model where parameters have physiological meaning or a differential (or difference) equation-based model where parameters do not have physiological meaning (e.g. time series models).

12. The system of claim 1, wherein the kidney health score is calculated based on a trained computation/prediction algorithm, the training performed by a learning algorithm, the learning algorithm trained based on health data from a plurality of patients, the health data including data related to healthy patients and data related to patients having the predicted kidney condition.

13. The system of claim 12, wherein the learning algorithm includes a mathematical process to update weights, coefficients, biases, and/or parameters of a nonlinear mapping function of the input values to one or more output values, the one or more output values including the kidney health score.

14. The system of claim 13, wherein the learning algorithm comprises an optimization algorithm.

15. The system of claim 12, wherein the prediction module is configured to update the trained computation/prediction algorithm based on new health data from a plurality of patients, the plurality of patients selected over a selected range of time from at least one of a selected care unit or facility, a selected geographical location, and a selected subset of a patient population.

16. The system of claim 1, further comprising a post-processing module configured to perform at least one of: storing the predicted kidney condition and the kidney health score in a results database, and performing an inference using the kidney health score to provide an advisory to a user.

17. The system of claim 16, wherein the advisory is at least one of a diagnostic protocol, a therapeutic protocol and an adjustment to a therapy.

18. The system of claim 1, wherein the input module is configured to establish communication with a medical device and/or system and receive input data therefrom.

19. The system of claim 1, further comprising a pre-processing module including a data acquisition system configured to perform at least one of parsing, interpreting, transforming, descaling, normalizing, unit conversion, storing, hosting, plotting, and sending to a prediction module.

20. The system of claim 1, wherein the predicted kidney condition is at least one of an acute kidney injury (AKI), reversible kidney damage, an intrinsic kidney disease, an extrinsic kidney disease, a pre-renal condition, an intrarenal condition, and a post-renal condition and is represented by at least one of a predicted stage, a severity, a percentage, and a probability of the kidney condition.

21. The system of claim 1, wherein the system is configured to administer a therapeutic modality or modify an existing treatment based on one or more output values from the prediction module, wherein the system effectuates the treatment or amelioration of at least one symptom of the predicted kidney condition.

22. A method of assessing kidney health, the method comprising:
receiving, by an input module, input values related to kidney function of a patient;
predicting, by a prediction module comprising a computation algorithm and/or a model, a kidney condition and calculating a kidney health score related to at least one of a severity and a probability of the predicted kidney condition by a prediction module, the kidney health score calculated based on the one or more input values; and
presenting, by an output module, the predicted kidney condition and the kidney health score to a medical professional.
